(19) United States
(12) Patent Application Publication
(10) Pub. No.: US 2004/0072138 A1
(43) Pub. Date: Apr. 15, 2004

(54) Attenuation of ischemia/reperfusion injury

(75) Inventor: Inderjit Singh, Mount Pleasant, SC (US)

Correspondence Address: FULBRIGHT & JAWORSKI L.L.P., 600 CONGRESS AVE., SUITE 2400, AUSTIN, TX, 78701, US

(73) Assignee: Medical University of South Carolina

(21) Appl. No.: 10/429,231

(22) Filed: May 2, 2003

Related U.S. Application Data
(63) Continuation-in-part of application No. 10/273,557, filed on Oct. 18, 2002.
Application No. 10/273,557 is a division of application No. 09/579,791, filed on May 25, 2000, now Pat. No. 6,511,800.
Application No. 09/579,791 is a continuation of application No. PCT/US98/25360, filed on Nov. 25, 1998.
(60) Provisional application No. 60/066,839, filed on Nov. 25, 1997.

(51) Int. Cl. A01N 1/00; A01N 1/02; A61K 31/7056; A61K 31/198

(52) U.S. Cl. 435/1.1; 514/43; 514/562

(57) ABSTRACT

This current invention includes compositions and methods of nitric oxide synthase inhibitors to treat or reduce ischemia/reperfusion injury in a patient. More specifically, the invention relates to a combinational therapy of 5-Aminoimidazole-4-carboxamide-1-B-D-ribonucleoside (AICAR) and N-acetyl cysteine (NAC) to attenuate ischemia/reperfusion injury to a transplanted organ.

This application is a continuation-in-part application of co-pending U.S. application Ser. No. 10/273,557 filed Oct. 18, 2002, which is a divisional application of U.S. application Ser. No. 09/579,791 filed May 25, 2000, now issued as U.S. Ser. No. 6,511,800, which is a continuation of prior international Application No. PCT/US98/25360 filed Nov. 25, 1998, which claims priority to U.S. Provisional Application Serial No. 60/066,839, filed Nov. 25, 1997. The entire text of the foregoing applications are specifically incorporated herein by reference without disclaimer.

### BACKGROUND OF THE INVENTION

1. Field of the Invention

The present invention relates to compositions for and methods of treating or reducing ischemia/reperfusion injury in organs.

2. Description of Related Art

Ischemia, the lack of oxygen to an organ, rapidly sets into motion a complex series of events that affect the structure and function of virtually every organelle and subcellular system of the affected cells. Ischemia/reperfusion injury leads to production of excessive amounts of reactive oxygen species (ROS) and reactive nitrogen species (RNS) causing oxidative stress which results in alterations in mitochondrial oxidative phosphorylation, depletion of ATP, an increase in intracellular calcium and activation of protein kinases, phosphatases, proteases, lipases and nucleases leading to loss of cellular function/integrity. Many studies show that an inflammatory response induced by ischemia followed by reperfusion is largely responsible for tissue damage (Vedder et al., 1990; Takada et al., 1997). The acute inflammatory response initiated by ischemia/reperfusion is characterized by the induction of a proinflammatory cytokine cascade (Herskowitz et al., 1990). Previous studies have shown that N-acetyl cysteine (NAC) inhibits the induction of pro-inflammatory cytokines and iNOS and production of NO (Pahan et al., 1998) and also blocks the TNF-α induced apoptotic cell death (Singh et al., 1998).

In other studies, N-acetyl cysteine (Koeppel et al. 1996), nitric oxide donors (Lopez-Neblina et al., 1996) and inhibitors of endothelin-1 converting enzyme (Vemulapalli et al., 1993; Bird et al., 1995) have been tested as therapeutic agents against ischemia/reperfusion injury with limited success. The inventor has shown the protective effect of combination therapy of N-acetyl cysteine, sodium nitroprusside and phosphoramidon against ischemia/reperfusion injury in rat kidney after 90 min of normothermic ischemia (Dobashi et al., 2002) and also in dog kidney (Sekhon et al., 2003b).

5-amino-4 imidazole carboxamide riboside (AICAR) has been shown to afford sustained protection against myocardial ischemia-reperfusion injury (Alkhulaifi and Pugsley, 1995; Galinanes et al., 1995) where it is thought to increase regional blood flow by increasing local adenosine concentration. It can inhibit neutrophil activation (Mathew et al., 1995) and suppress platelet aggregation (Bullough et al., 1994).

Ischemic/reperfusion injury in organ transplantation is especially problematic because the harvested organ is removed from the body, isolated from a blood source, and thereby deprived of oxygen and nutrients for an extended period of time (U.S. Pat. No. 5,912,019). In fact, one of the most critical problems in transplantation today is the relatively high incidence of delayed graft function (DGF) due to acute tubular necrosis (ATN) after surgery.

For example, DFG affects 20-35% of kidney transplants in many transplant centers and is the most common complication of the immediate postoperative period in renal transplantation. Although the incidence and definition of DGF vary among transplant centers, the consequences are uniform: prolonged hospital stay, additional invasive procedures, and additional cost to the patient and health-care system. DGF impacts both the individual patient and the infrastructure for organ procurement and sharing because of the drain it places on the available organ supply. DFG also elevates the risk of early acute rejection episodes and increases early graft loss from chronic rejection (Koning et al., 1997; Ojo et al., 1997; Matas et al., 2000).

Using current preservation methods, cold ischemia incurred during organ preservation has been identified as a major risk factor in causing delayed graft function after transplant. For kidneys, cold ischemia times in excess of 24 hr are associated with a significantly increased risk of delayed graft function (Koning et al., 1997; Ojo et al., 1997; Tejani et al., 1997).

Effective cold preservation of kidneys was first successfully achieved by Belzer et.al, (1967), using machine perfusion and a solution derived from cryoprecipitated plasma (Belzer et.al, (1967). This was followed by the introduction of simple cold-storage methods by Collins et.al, (1969), using a cold crystalloid solution. Since these early successes with kidney, preservation solutions have evolved into entirely synthetic defined media designed to prevent cold ischemic injury by suppression of cell swelling and provision of metabolic support. The first such synthetic solution was the University of Wisconsin (UW) lactobionate-based solution, described by Wahlberg et. al, (1986) and subsequently proven effective in pancreas (Wahlberg et al., 1987) and liver (Jamieson et al., 1988). UW solution continues today to be considered the gold standard by which new cold-storage preservation solutions are evaluated.

The evolution of the UW solution from its original blood product based composition to its current totally synthetic serum-free formulation has resulted in significant improvements in the quality and duration of feasible organ preservation (Ploeg et al., 1988). However, clinical data on delayed graft function in kidneys clearly demonstrate that this solution is not completely successful in preventing cold ischemic graft injury, highlighting that the mechanisms of injury in cold-stored organs remain poorly understood, and that further progress in developing methods for suppression of cold ischemic injury is needed.

Therefore, there exists a need for better means of treating or reducing ischemic/reperfusion injury to tissues and organs caused by organ transplantation.

### SUMMARY OF THE INVENTION

The present invention overcomes the deficiencies in the art by providing a surprising and effective combination of AICAR and NAC to treat or reduce ischemic/reperfusion injury in tissue and organs.

In one aspect of the present invention, the inventors contemplate pharmaceutically acceptable compositions and methods for reducing ischemic/reperfusion injury to an organ in a patient. The pharmaceutically acceptable composition comprises 5-aminoimmidazole-4-carboxamide ribonucleoside (AICAR) and N-acetyl cysteine (NAC). The methods include administering a therapeutically effective amount of AICAR and NAC to the patient.

It is further contemplated that the combination of AICAR and NAC can also reduce the risk of and/or treat organ failure caused by ischemic/reperfusion injury and reduce and/or treat delayed graft function of a transplanted organ in a patient. In some aspects, delayed graft function is caused by acute tubular necrosis.

In some instances, the organ will be transplanted into the patient. In these situations, AICAR can be administered to the patient before, during and/or after the organ is transplanted into the patient. Similarly, NAC can be administered to the patient before, during and/or after the organ is transplanted into the patient.

In other aspects of the present invention, AICAR can be administered to the patient, before, during or after NAC is administered to the patient.

AICAR can be administered to the patient from about 30 min to about 90 min before the organ is transplanted. Similarly, NAC can be administered to the patient from about 30 min to about 90 min before the organ is transplanted. It should be noted that these compounds can be administered at any time between 30 to 90 min, including but not limited to 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88 or 89 min, or any fraction in between. It is also contemplated, that the compositions could be administered at times outside the range of 30 to 90 min.

In preferred aspects, the organ is a heart, kidney, liver, pancreas or brain. In more preferred embodiments of the present invention, the organ is a kidney. As used throughout this application, “organ” means a part of a mammalian subject composed of several tissues and adapted to perform a specific function or functions. Representative organs include, but are not limited to, the heart, kidney, liver, pancreas and brain.

The inventor also contemplates administering from about 1 to about 100 mg/kg body weight of AICAR to a patient. Similarly, it is contemplated that from about 1 to about 200 mg/kg body weight of NAC can be administered to the patient. The amount of AICAR and/or NAC administered to a patient may vary or fall out side of the ranges given above. As discussed in other section of this application, the amount of AICAR and/or NAC administered to the patient can vary.

As used throughout the specification and/or claims, the following terms mean:

“Derivatives” refers to chemically modified compounds inhibitors or stimulators that still retain the desired effects on property(s) of nitric oxide production or pro inflammatory gene, protein, and/or activity induction or suppression. Such derivatives may have the addition, removal, or substitution of one or more chemical moieties on the parent molecule. Such moieties may include, but are not limited to, an element such as a hydrogen or a halide, or a molecular group such as a methyl group. Such a derivative may be prepared by any method known to those of skill in the art. The properties of such derivatives may be assayed for their desired properties by any means described herein or known to those of skill in the art.

“Analogs” include structural equivalents or mimetics, described further in the detailed description.

A “patient” or “subject”, as used herein, may be an animal. Preferred animals are mammals, including but not limited to humans, pigs, cats, dogs, rodents, horses, cattle, sheep, goats and cows. Preferred patients and subjects are humans.

“Inhibitors” refers to such compounds or agents that produce any measurable decrease in the activity, production, or secretion of a protein or biological compound, or the translation of mRNA, in, or in the case of secretion, from, a cell.

“Reducing ischemic/reperfusion injury” means any measurable decrease or reversal of damage to organs that are stored or transplanted into a patient. Similarly, “reducing” means any measurable decrease or complete inhibition of damage to organs that are stored or transplanted into a patient.

The words “a” and “an,” as used in this specification, including the claims, denotes “one or more.” Specifically, the use of “comprising,” “having,” or other open language in claims that claim a combination or method employing “an object,” denotes that “one or more of the object” may be employed in the claimed method or combination.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Effect of AICAR and NAC on the life span of dogs and kidney function. FIG. 1A Effect of AICAR and NAC on survival of dogs after 48 hr of cold ischemia to the kidneys. The dogs in the ischemia alone group had to be euthanized to post-op day 1 to post-op day 4 while the treated animals had 100% survival. FIG. 1B Effect of AICAR and NAC on serum creatinine levels in dogs after 48 hr of cold ischemia to kidney. Serum creatinine was measured using a creatinine kit (procedure no. 555) obtained from Sigma Diagnostic Co., St. Louis, Mo. The values are expressed as mean +/− SEM for n=5 (untreated) and n=7 (treated). FIG. 1C Effect of AICAR and NAC on blood urea nitrogen levels in dogs after 48 hr of cold ischemia to kidney. BUN was measured using a blood urea nitrogen kit (procedure no. 535) obtained from Sigma Diagnostic Co., St. Louis, Mo. The values are expressed as mean SEM for n=5 (untreated) and n=7 (treated).

FIG. 2. Effect of AICAR and NAC on the morphology of renal tissue following ischemia/reperfusion injury is shown in the light microscopic pictures of kidney tissues from control (A). Untreated (B) and AICAR and NAC treated (C) (representative of 20 photomicrographs; H & E magnification 200×). Tissue sections were stained for morphologic evaluation as discussed in Example 1.

FIG. 3. Effect of AICAR and NAC on the expression of TNF-α in kidney tissue following ischemic/reperfusion injury. Tissue sections were immunostained with antibodies against TNF-α as described in Example 1 (representative of 20 photomicrographs; magnification 400×). (A) control kidney. (B) Untreated kidney at 1 day of reperfusion. (C) Treated kidney at 1 day of reperfusion. (D) Treated kidney at 14 days of reperfusion.

FIG. 4. Effect of AICAR and NAC on the expression of IFN-γ in kidney tissues following ischemia/reperfusion injury. Tissue sections were immunostained with antibody against IFN-γ as described in Example 1 (representative of 20 photomicrographs; magnification 400×). (A) control kidney. (B) Untreated kidney at 1 day of reperfusion. (C) Treated kidney at 1 day of reperfusion. (D) Treated kidney at 14 days of reperfusion.

FIG. 5. Effect of AICAR and NAC on the expression of iNOS in kidney tissue following ischemia/reperfusion injury. Tissue sections were immunostained with antibody against iNOS as described in Example 1 (representative of 20 photomicrographs; H & E magnification 400×). (A) control kidney. (B) Untreated kidney at 1 day of reperfusion. (C) Treated kidney at 1 day of reperfusion. (D) Treated kidney at 14 days of reperfusion.

FIG. 6. Effect of AICAR and NAC on apoptosis (tunel assay) in kidney tissue following ischemia/reperfusion injury. Tissue sections were immunostained for detection of apoptosis as described in Example 1 (representative of 20 photomicrographs; magnification 200×). (A) control kidney. (B) Untreated kidney at 1 day of reperfusion. (C) Treated kidney at 1 day of reperfusion. (D) Treated kidney at 14 days of reperfusion.

### DETAILED DESCRIPTION OF THE INVENTION

Ischemia, the lack of blood to an organ rapidly sets into motion a complex series of events that affect the structure and function of virtually every organelle and subcellular system of the affected cells. Ischemic insult is very often the end point of many disease processes leading to significant morbidity and mortality. Ischemic/reperfusion injury is especially problematic in organ transplantation because the harvested organ is removed from the body, isolated from a blood source, and thereby deprived of oxygen and nutrients for an extended period of time (U.S. Pat. No. 5,912,019).

Previous studies have shown that nitric oxide (NO), a diffusible free radical, appears to have both neurotoxic and neuroprotective effects and may have a role in the pathogenesis of ischemia and traumatic injuries associated with infiltrating macrophages and the production of proinflamatory cytokines (Mitrovic et al., 1994; Bo et al., 1994; Merrill et al., 1993; Dawson et al., 1991, Kopranski et al., 1993; Bonfoco et al., 1995).

The inventors have discovered that inhibitors of nitric oxide synthase, the enzyme that produces NO, are useful for treating or reducing ischemic/reperfusion injury in organs and tissue. More specifically, the inventors have discovered that the combination of AICAR and NAC can be used to reduce and/or treat ischemia/reperfusion injury in organs and tissue.

### 1. Nitric Oxide Synthase

NO is synthesized from L-arginine by the enzyme nitric oxide synthase (NOS) (Nathan, 1992). Nitric oxide synthases are classified into two groups. One type, constitutively expressed (cNOS) in several cell types (e.g., neurons, endothelial cells), is regulated predominantly at the post-transcriptional level by calmodulin in a calcium dependent manner (Nathan, 1992; Jaffrey et al., 1995). In contrast, the inducible form (iNOS), synthesized de novo in response to different stimuli in various cell types including macrophages, hepatocytes, myocytes, neutrophils, endothelial and messangial cells, is independent of calcium.

### 2. Inhibitors of Nitric Oxide Synthase

Inhibitors of nitric oxide synthase include, but not limited to, lovastatin, mevastatin, FPT inhibitor II, forskolin, rolipram, phenylacetate (NaPA), N-acetyl cysteine (NAC), pyrolidine dithiocarbamate (PDTC), 4-phenylbutyrate (4PBA), 5-aminoimmidazole-4-carboxamide ribonucleoside (AICAR), theophylline, papaverine, cAMP, 8-bromo-cAMP, (S)-cAMP, and salts, analogs, or derivatives thereof.

i. 5-Amino 4-Imidazolecarboxamide Ribotide (AICAR)

AICAR has been shown to reduce ischemic complications during coronary artery bypass graft surgery (Mangano, 1995; Menasche, 1995). The inventor has shown that activation of AMP-activated protein kinase (AMPK) by AICAR down regulated the LPS mediated induction of pro-inflammatory cytokines, iNOS and NO production in rat primary astrocytes, microglia and peritoneal macrophages (communicated). AMPK has been recently reported to have a protective function during glucose deprivation in neurons (Culmsee et al., 2001) and it has been shown to protect astrocytes (Blazquez et al., 2001) and thymocytes (Stefanelli et al., 1998) from apoptosis and necrosis. These observations indicate that AMPK may be target molecule for anti-inflammatory drugs such as AICAR. AICAR has been previously used as a drug for treating Lesch-Nyhan Syndrome at a relatively high dose (100 mg/kg body weight) safely and without any side effects (Page et al., 1994).

ii. NAC

NAC is an antioxidant that reacts with hydroxyl radical and hypochloric acid but is poorly reactive with hydrogen peroxide and the superoxide radical. NAC has been used in a variety of disease states like acetaminophen toxicity, pulmonary oxygen toxicity and human immunodeficiency virus infection. The inventor has shown the protective effect of pre-administration of NAC on focal cerebral ischemia in rats (Sekhon et al., 2003a)

Induction of iNOS under inflammatory condition leads to the production of large amount of NO for longer period of time. The toxic effect of NO may be attributed to peroxynitrite (ONOO), which is a reaction product of NO with oxygen. NAC has been shown to be beneficial in ischemic brain injury by inhibiting NO production. NAC has efficiently blocked the iNOS expression.

Reactive oxygen species have been shown to cause direct DNA damage (Hagar et al., 1996). NAC has reduced ischemia-induced DNA fragmentation. Previous studies from the inventor have shown that NAC also inhibits the induction of pro-inflammatory cytokines (Pahan et al., 1998) and also blocks the TNF-α induced apoptotic cell death in cultured brain cells (Singh et al., 1998).

### 3. Second Generation Inhibitors

In addition to the nitric oxide synthase inhibitory compounds initially identified, the inventor also contemplates that other sterically similar compounds may be formulated to mimic the key portions of the structure of these inhibitors. Such compounds may be used in the same manner as the nitric oxide synthase inhibitors identified herein.

The generation of further structural equivalents or mimetics may be achieved by the techniques of modeling and chemical design known to those of skill in the art. The art of computer-based chemical modeling is now well known. Using such methods, a chemical compounds acting in a similar manner as those nitric oxide synthase inhibitors identified herein can be designed and synthesized. It will be understood that all such sterically similar constructs and second generation molecules fall within the scope of the present invention.

### 4. Optimization in Therapy

A compound identified as having the ability to reduce ischemic/reperfusion injury in a subject can be assayed by its optimum therapeutic dosage alone or in combination with another such compound. Such assays are well known to those of skill in the art, and include tissue culture or animal models for various disorders that are treatable with such agents.

Examples of such assays include those described herein and in U.S. Pat. No. 5,696,109. For instance, an assay to determine the therapeutic potential of molecules in brain ischemia (stroke) evaluates an agent's ability to prevent irreversible damage induced by an anoxic episode in brain slices maintained under physiological conditions. An animal model of Parkinson's disease involving iatrogenic hydroxyl radical generation by the neurotoxin MPTP (Chiueh et al., 1992, incorporated herein by reference) may be used to evaluate the protective effects of iNOS or pro-inflammatory cytokine induction inhibitors. The neurotoxin, MPTP, has been shown to lead to the degeneration of dopaminergic neurons in the brain, thus providing a good model of experimentally induced Parkinson's disease (e.g., iatrogenic toxicity). An animal model of ischemia and reperfusion damage is described using isolated iron-overloaded rat hearts to measure the protective or therapeutic benefits of an agent. Briefly, rats receive an intramuscular injection of an iron-dextran solution to achieve a significant iron overload in cardiac tissue. Heart are then isolated and then subjected to total global normothermic ischemia, followed by reperfusion with the perfusion medium used initially. During this reperfusion, heart rate, and diastolic and systolic pressures were monitored. Cardiac tissue samples undergo the electron microscopy evaluation to measure damage to mitochondria such as swelling and membrane rupture, and cell necrosis. Comparison of measured cardiac function and cellular structural damage with or without the agent or iron-overloading after ischemia/reoxygenation is used to determine the therapeutic effectiveness of the agent.

### 5. Combination Therapy

In order to increase the effectiveness of the ischemic/reperfusion injury therapy of the present invention, it may be desirable to combine the present compositions/combination therapy with other agents and methods effective in the treatment ischemic/reperfusion injury. Such agents include, but are not limited to, anti-inflammatory agents, particularly non-steroidal anti-inflammatory drugs (NSAIDs), vasodilator prostaglandins including prostacyclin and prostaglandin E sub 1, cancer chemotherapeutic agents including cisplatin, NO donors or NO inhalation therapy, or PAF—receptor antagonists. Other possible agents that can be used in combination with the present invention include nitro-glutathione and curcumin.

Combinations of nitric oxide synthase inhibitors are provided in a combined amount effective to confer a therapeutic benefit to a person suffering an ischemic/reperfusion injury. This process may involve administering the combination of AICAR and NAC and another agent(s) to the subject at the same time, for example, using a single composition or pharmacological formulation that includes both agents, or using two distinct compositions or formulations given at the same time, wherein one composition includes AICAR administered before, during or after NAC and the other includes the second agent(s). Alternatively, the second agent therapy may precede or follow the AICAR/NAC combination treatment by intervals ranging from min to weeks.

The exact schedule of treatment with the AICRA/NAC combination therapy and the second agent is determined in large part by the pharmacokinetic or pharmacodynamic properties of AICAR, NAC and the second agents.

In embodiments where the other agent and the AICAR/NAC combination therapy are administered separately to the subject, one may wish that a significant period of time did not expire between the time of each delivery, such that the second agent and the AICAR/NAC combination therapy would be able to exert an advantageously combined effect on the subject. In such instances, it is contemplated that one may administer to the subject with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, the AICAR/NAC combination therapy is “A” and the second agent is “B”:

| | | | | | | | |
|---|---|---|---|---|---|---|---|
| A/B/A | B/A/B | B/B/A | A/A/B | A/B/B | B/A/A | A/B/B/B | B/A/B/B |
| B/B/B/A | B/B/A/B | A/A/B/B | A/B/A/B | A/B/B/A | B/B/A/A | | |
| B/A/B/A | B/A/A/B | A/A/A/B | B/A/A/A | A/B/A/A | A/A/B/A | | |

### 6. Pharmaceutical Compositions

Pharmaceutical compositions of the present invention comprise AICAR and NAC. The phrases “pharmaceutical or pharmacologically acceptable” refers to molecular entities and compositions that do not produce an adverse, allergic or other untoward reaction when administered to an animal, such as, for example, a human. The preparation of a pharmaceutical composition comprising AICAR and NAC will be known to those of skill in the art in light of the present disclosure, as exemplified by Remington's Pharmaceutical Sciences, 18th Ed. Mack Printing Company, 1990. Moreover, for animal (e.g., human) administration, it will be understood that preparations should meet sterility, pyrogenicity, general safety and purity standards as required by FDA Office of Biological Standards.

“Therapeutically effective amounts” are those amounts effective to produce beneficial results, particularly with respect to cancer treatment, in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). Except insofar as any conventional carrier is incompatible with the active ingredient, its use in the therapeutic or pharmaceutical compositions is contemplated.

The compositions of the present invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (Remington's, 1990).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The compositions may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments, the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments, the compositions are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments, an oral composition may comprise one or more binders, excipients, disintegration agents, lubricants, flavoring agents, and combinations thereof.

In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition should be stable under the conditions of manufacture and storage, and preserved against the contaminating action of microorganisms, such as bacteria and fungi. It will be appreciated that exotoxin contamination should be kept minimally at a safe level, for example, less that 0.5 ng/mg protein.

### 7. Kits

In one aspect, the invention provides a therapeutic kit comprising, in suitable container means, a therapeutically-effective amount of one or more nitric oxide synthase inhibitors. In preferred embodiments, the kit will include a composition comprising AICAR and NAC.

The container means will generally include at least one vial, test tube, flask, bottle, syringe or other container means, into which the composition(s) may be placed, preferably, suitably allocated. The kits may also comprise a second/third container means for containing a sterile, pharmaceutically acceptable buffer or other diluent.

The kits of the present invention will also typically include a means for containing the vials in close confinement for commercial sale, such as, e.g., injection or blow-molded plastic containers into which the desired vials are retained. Alternatively, the vials may be prepared in such a way as to permit direct introduction of the composition into an intravenous drug delivery system.

The following examples are included to demonstrate new and inventive methods of the inventor and preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials and Methods

Animals: Male mongrel dogs weighting 15-20 kg (Marshal Farms, N.Y.) were used in this study. These animals were acclimatized and maintained on a standard diet for 1 week before the surgical procedure. All animals received humane care in compliance with the Medical University of South Carolina's guidelines and the National Research Council's criteria for humane care as outline in “Guide for the Care and Use of Laboratory Animals”.

Antibodies and Kits: Mouse monoclonal TNF-α antibody (SC-7317) and goat polyclonal IFN-γ antibody (SC-1377) were obtained from Santa Cruz Biotechnology, Inc., Santa Cruz, Calif. Rabbit polyclonal iNOS antibody (N32030-050) was obtained from Transduction Laboratories, San Diego, Calif. Apop tag plus peroxidase in situ apoptosis detection kit (S7101) was obtained from Intergen Company, NY. Creatinine (procedure no. 555) and blood urea nitrogen (procedure no. 535) kits were obtained from Sigma Diagnostics Co. St. Louis, Mo.

Experimental Groups and Operative Technique: After overnight fast, the animals were preanesthetized with subcutaneous buprenorphin (0.02 mg/kg body weight) ½ hr prior to sedation. An intravenous catheter was placed in the cephalic vein and antibiotic prophylaxis Cefazolin sodium 500 mg was given. The dogs were sedated with Pentothal sodium 3 mg/kg body weight and then maintained during the procedure with isoflurane (0.5-2.0% in O₂. An epidural injection of morphine (0.1 mg/kg body weight) was given to obtain prolonged post-operative analgesia. The abdominal skin was sterilized with povidine-iodine. A midline incision was given. The left kidney was exposed, carefully dissected from perirenal tissue and fat and was removed following the ligation of ureter, renal artery and vein with 3-0 silk sutures. The kidney was placed on ice and the renal artery was cannulated to flush the kidney with 150-250 ml of cold University of Wisconsin solution. The flushed kidney was preserved in the flush-out solution in the isolation bags and kept at 4° c. The midline incision was sutured in three layers; muscle peritoneum with 2-0 vicryl, subcutaneous fascia with 3-0 vicryl and skin layer with 3-0 nylon.

After 48 hr the same dog was anesthetized, catheterized with a urinary catheter and opened through midline again. The right kidney was exposed, isolated and removed following ligation of right renal pedicle with 3-0 silk. Inferior vena cava and the abdominal aorta were isolated just above the aortic bifurcation in the lumbo-sacral area. The preserved kidney was taken out of ice and the renal artery and vein were anastomosed to abdominal aorta and inferior vena cava respectively. Submuscular tunneling technique was used for ureteral anastomosis into the urinary bladder. Kidney was reperfused and the blood flow was monitored visually to and from the kidney. Abdomen was closed as described earlier. The animals were divided into the following groups:

1. Untreated group. (n=5)—The above procedure was followed but no compound was tested.

2. Treatment group. (n=7)—The above procedure was followed and the following compounds were used as follows:

(i) AICAR at 50 mg/kg body wt. in a slow intravenous infusion over 30 min starting 30 min before left nephrectomy and 30 min before implantation of the preserved kidney.

(ii) NAC at 100 mg/kg body weight intravenously one hour before nephrectomy and one hour before implant.

(iii) AICAR and NAC were also added to the flushing solution (UW solution) at 100 mg and 200 mg per kilo weight of kidney respectively.

The animals were allowed food and water after recovering from anesthesia. Blood samples were drawn post-operatively at different time intervals and serum creatinine and blood urea nitrogen were measured for up to 14 days of reperfusion. The animals were sacrificed at different time points of reperfusion for morphological and immunohistochemical studies on the harvested kidney.

Light Microscopic Evaluation of Renal Tissue Sections: Light microscopy was done to evaluate the morphology of kidney tissue from different experimental groups. Kidneys were fixed in 10% buffered formalin (Stephens Scientific, Riverdale, N.J.). The tissues were embedded in paraffin and sectioned at 4 mm thickness. Sections were then stained with hematoxylin and eosin (H & E) stain to evaluation morphological changes.

Immunoshistochemistry: Since proinflammatory cytokines (TNF-α, IFN-γ) and iNOS play an important role in the pathogenesis of kidney ischemia/reperfusion injury, the expression of these cytokines was determined by immunohistochemical analysis using highly specific antibodies. Paraffin embedded sections from the formalin fixed kidneys were stained for various cytokines (TNF-α, IFN-γ) and iNOS. In brief, the kidney sections were deparaffinized and sequentially rehydrated in graded alcohol and then immersed in phosphate buffered saline (PBS, pH 7.4). Slides were then microwaved for two minutes in antigen unmasking fluid (Vector Labs, Burlingame, Calif.), cooled and washed 3× for 2 min in PBS. Sections were immersed for 25 min in 3% hydrogen peroxide in distilled water to eliminate endogenous peroxidase activity, then blocked in immunohistochemical grade BSA (1% PBS) for 1 hr and diluted goat serum (Vector ABC Elite kit) for 30 min to reduce non-specific staining. Sections were incubated overnight with primary antibody (TNF-α, diluted in blocking buffer to 1:50, IFN-γ, diluted in blocking to 1:100 and iNOS, diluted in blocking buffer to 1:200) and then rinsed 3× for 6 min in PBS containing 0.1% Tween-20. Afterwards, iNOS was detected wit a rabbit polyclonal antibody and an avidin biotin HRP complex (Vectastain ABC-Elite kit, Vector Laboratories, Burlingame, Calif.) with diaminobenzidine (DAB) as a substrate. TNF-α and IFN-γ were detected with mouse monoclonal antibody and goat polyclonal antibody respectively (Santa Cruz Biotechnology, Inc., Santa Cruz, Calif.). Slides were then dehydrated through a graded series of alcohol and mounted in permount and cover slipped. All of the sections were analyzed using a Zeiss Olympus Microscope and images were captured using a Kontron digital camera controlled by Adobe Photoshop (Adobe Systems, Calif.).

Tunel Assay: Tunel assay was performed to detect apoptosis as a marker of all death due to ischemia/reperfusion injury. Renal tissue sections were deparaffinized and rehydrated through three changes of xylene and graded alcohol and washed in PBS for 5 min and then incubated in 20 μg/ml proteinase k for 15 min at room temperature. The apoptag plus peroxidase kit (Intergen Company) was used according to the manufacturer's instructions. Briefly, endogenous peroxidase activity in the kidney sections was blocked by incubation for 5 min with 3% H₂O₂ in phosphate buffered saline, followed by incubation for 10 seconds with equilibration buffer. The sections were then incubated for 60 min at 37° c. with terminal deoxynucleotidyl transferase (TdT) enzyme in reaction buffer. The reaction was termninated by incubation with stop buffer at room temperature. Sections were then incubated with peroxidase conjugated anti-digoxigenin antibody for 30 min at room temperature and the reaction was developed with DAB as substrate for 4 min at room temperature. Sections were counterstained with methyl green stain, dehydrated through a graded series of alcohol and mounted in permount for microscopy.

Statistical Analysis: Data are expressed as mean values±standard error of mean (SEM) for each number of experiments. Statistical analysis was evaluated by two-tailed t-test for unpaired observations. Differences at P<0.05 were taken as statistically significant.

### EXAMPLE 2

### Results

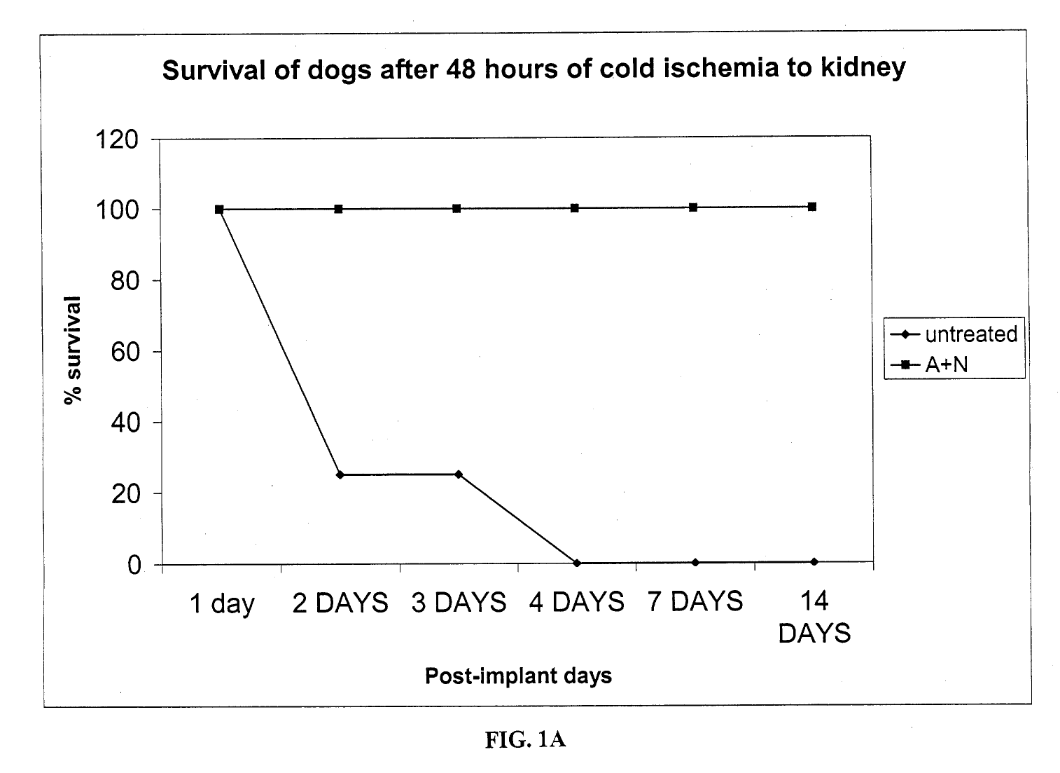

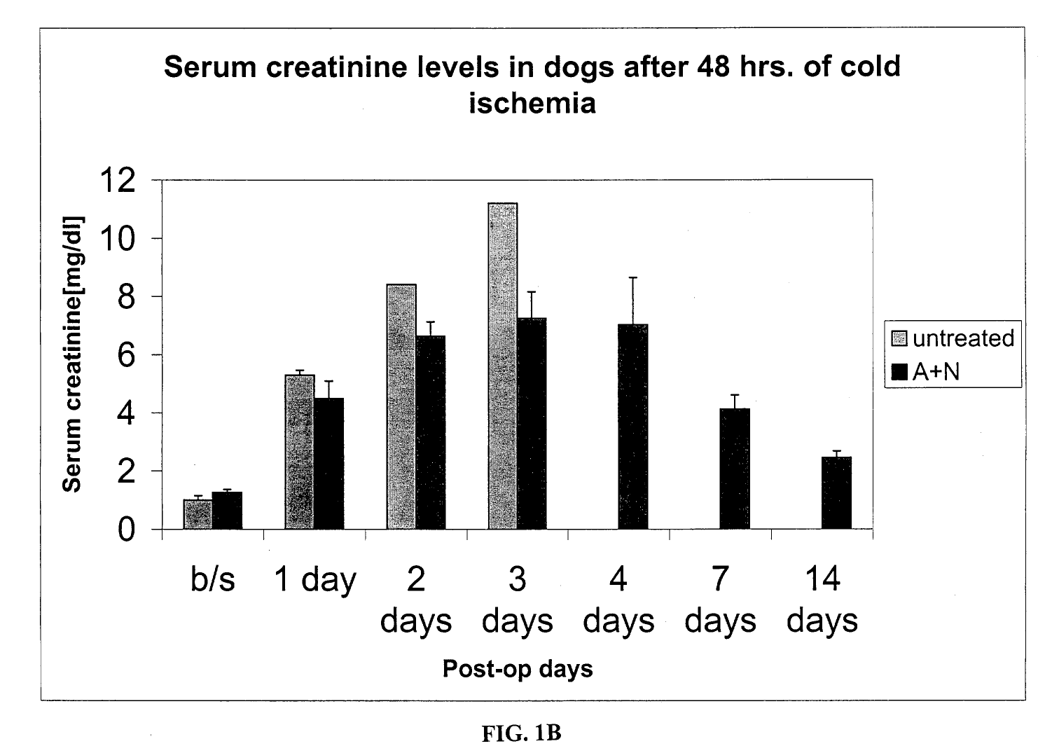

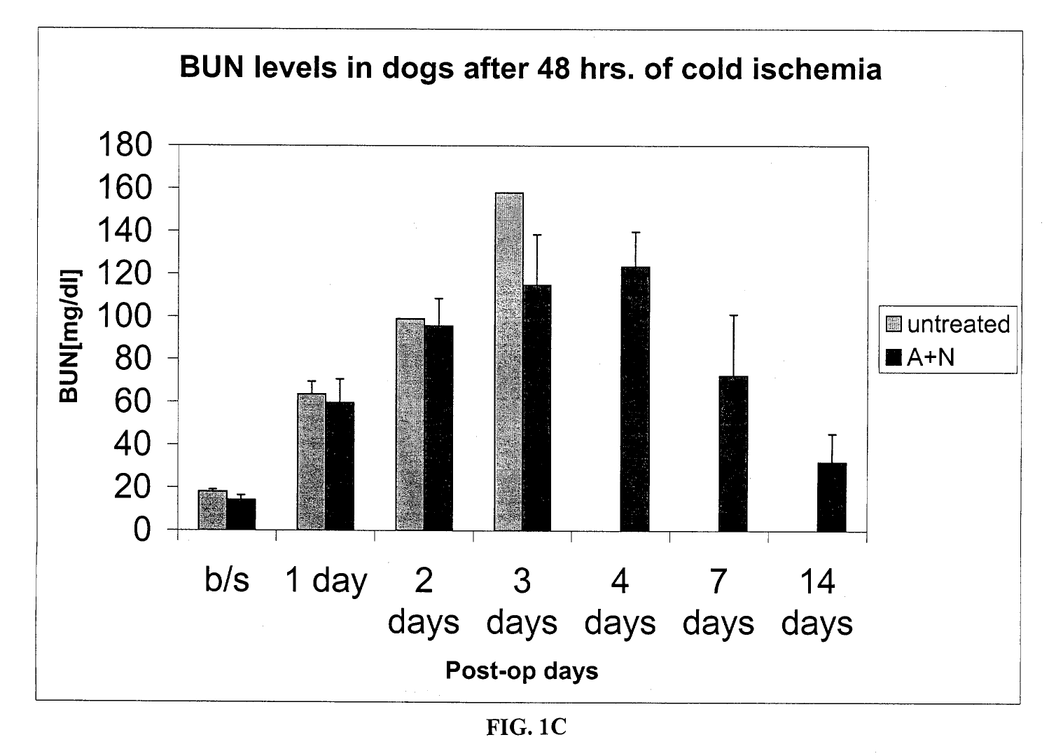

Treatment with AICAR+NAC increased the life span of the dogs and protected the functions of kidney exposed to ischemia/reperfusion injury (FIG. 1): The dogs in the ischemia group had to be euthanized at post-op day 1 to post-op day 4 because of poor clinical condition, severe azotemia and oliguria indicating an acute renal failure while the dogs in the AICAR+NAC treatment group were completely protected showing 100% survival (FIG. 1A). These dogs were euthanized after 14 days of reperfusion (the maximum time approved for the protocol by the IACUC, Medical University of South Carolina, Charleston, S.C.). Serum creatinine levels, measured as an index of kidney function, peaked to 11.2 mg/dl in the untreated group prior to euthanasia while in the treatment group, serum creatinine peaked to mean 7.26 mg/dl by day 3 of reperfusion (FIG. 1B). Blood urea nitrogen, measured as another index of kidney function, peaked to 158 mg/dl prior to euthanasia in the untreated group. BUN levels in the treated group peaked to mean 123.4 mg/dl by day 4 of reperfusion (FIG. 1C). These serum creatinine and BUN levels in the treated group came down to near normal after 14 days of reperfusion.

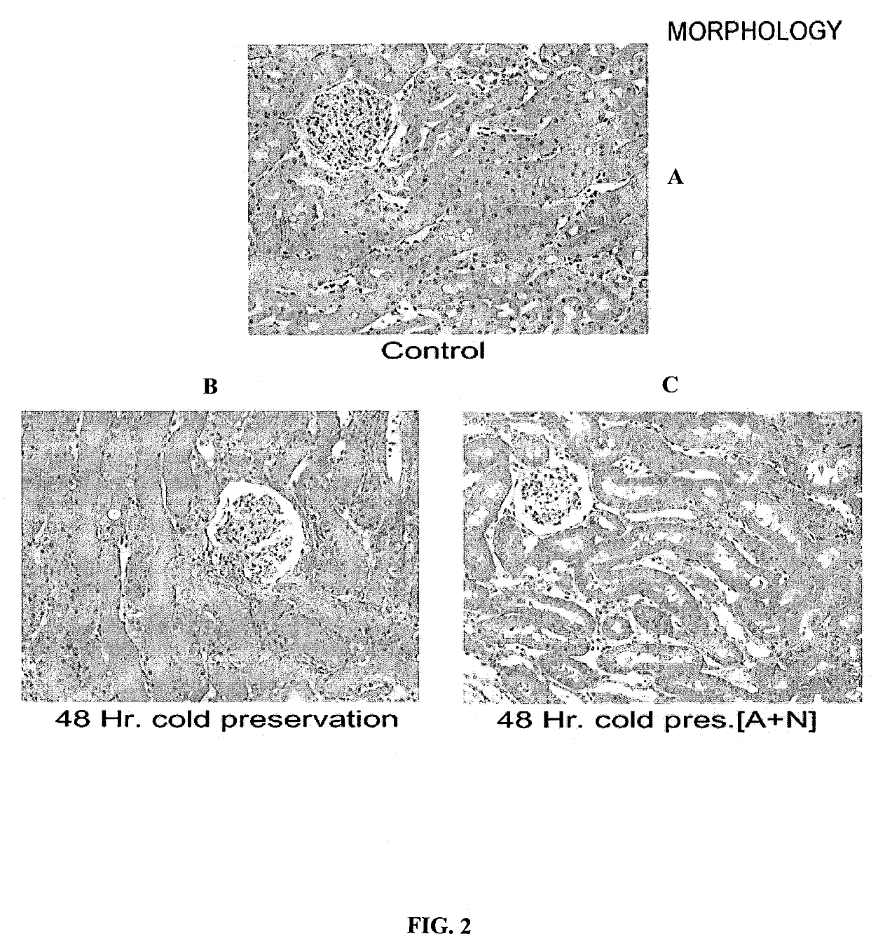

Light Microscopic Evaluation of the Kidney Sections for Morphological Changes: H & E stained histological sections of the ischemic kidney (FIG. 2B & FIG. 2C) showed the most prominent changes in the proximal tubules. The tubular lining cells shed into the tubular lumen leading to increased amounts of protein in the lumen. Some tubules showed complete loss of the lining cells, while others showed single cell necrosis with nuclear pyknosis and cytoplasmic eosinophilia. There was congestion of the tissue with foci of interstitial hemorrhage. The changes were more marked in the medulla. Sections of the treated kidney showed architectural and cytologic preservation. A few foci of dystrophic calcification were seen (FIG. 2D). Overall, the morphological features were similar to those seen in the control kidney (FIG. 2A).

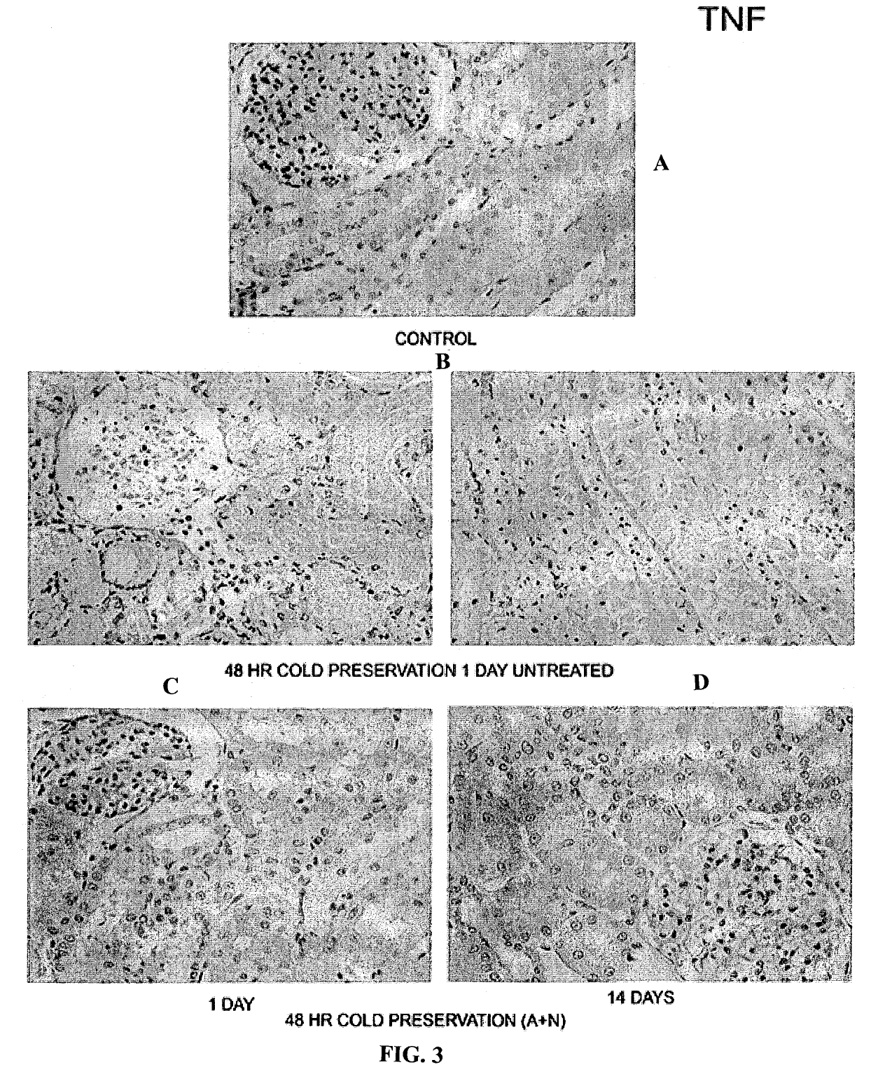

Immunohistochemical Localization of TNF-α: No TNF-α expression was detected in the control kidney (FIG. 3A). TNF-α was identified at day 1 of reperfusion, distributed in the shed proximal tubule cells in the ischemic kidneys (FIG. 3B). The treatment group showed a greatly reduced expression of TNF-α in kidney tissues at day 1 of reperfusion (FIG. 1C) while at 14 days of reperfusion, no TNF-α expression could be detected in the kidney sections from the AICAR+NAC treated animals (FIG. 3D).

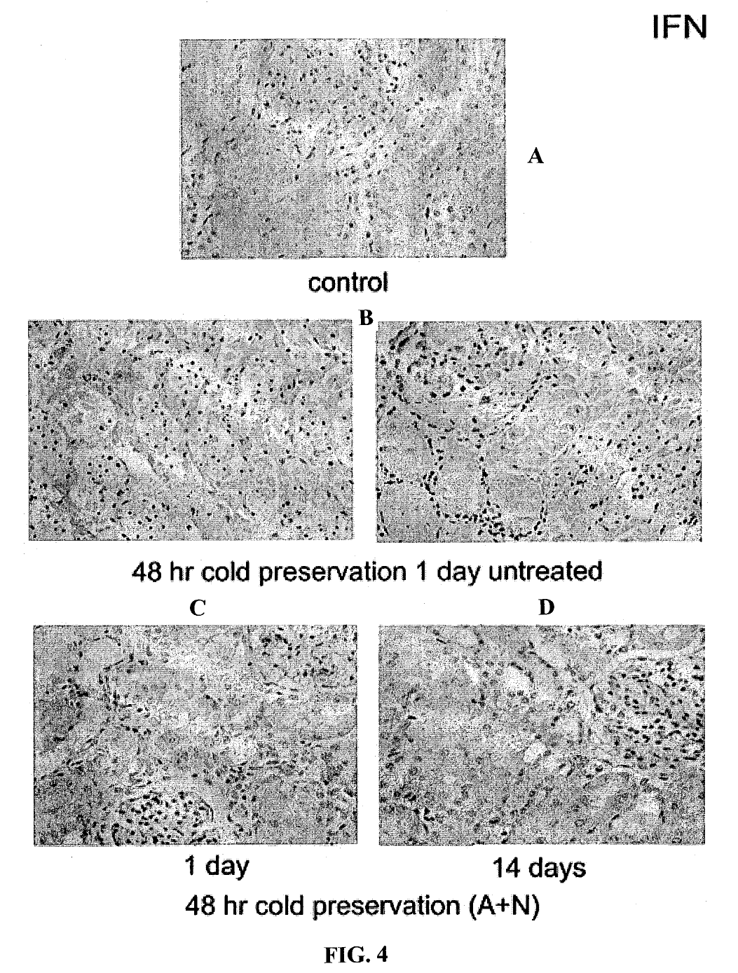

Immunohistochemical Localization of IFN-γ: No IFN-γ expression could be detected in control kidneys (FIG. 4A). IFN-γ expressed was detected in the ischemic kidneys at 1 day of reperfusion and distributed in the shed tubular epithelial in the tubular lumen (FIG. 4B). A reduced amount of IFN-γ expression was detected in the kidneys from treated group at 1 day of reperfusion (FIG. 4C). There was no IFN-γ expression in the kidney sections from AICAR+NAC treated animals at 14 days of reperfusion (FIG. 4D).

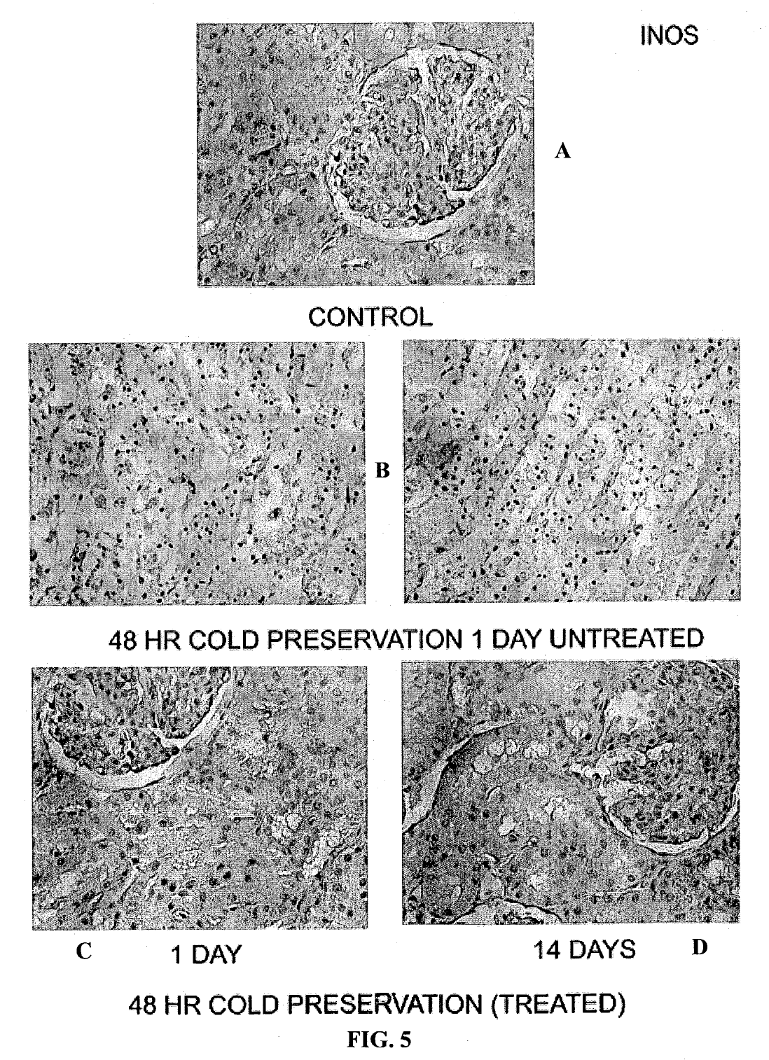

Immunohistochemical Localization of iNOS: No iNOS expression was seen in the control kidney sections (FIG. 5A). iNOS expression was detected in the ischemic kidney sections at 1 day of reperfusion and was found to be distributed in the shed tubular epithelial cells in the tubular lumen (FIG. 4B). A reduced amount of iNOS expression was seen in the AICAR+NAC treated kidney sections at 1 day of reperfusion (FIG. 4C) while no iNOS expression could be detected in the kidney sections from AICAR+NAC treatment group at 14 days of reperfusion (FIG. 4D).

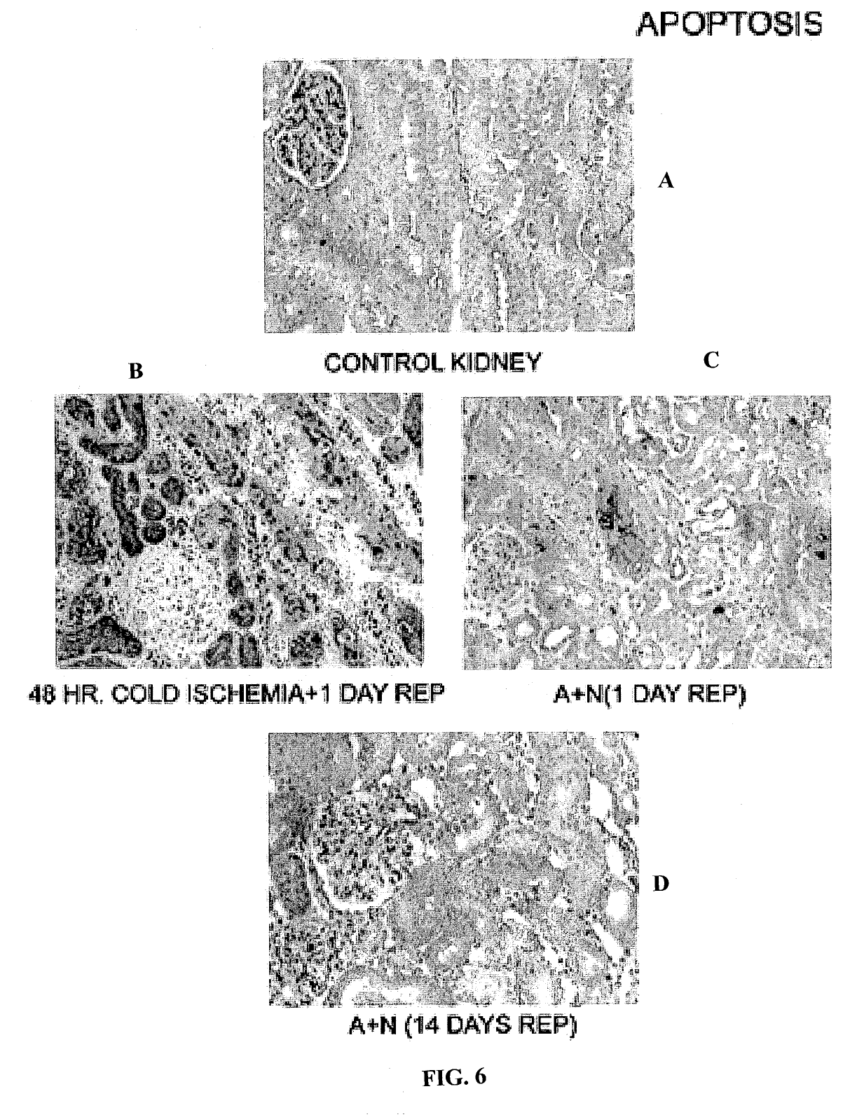

Immunohistochemical Localization of Apoptotic Cells (Tunel Assay): Examination of tunel-stained sections from control animals revealed very few apoptotic cells in the proximal convoluted tubules and glomerulus (FIG. 6A). While the untreated group showed and increased number of apoptotic cells (FIG. 6B) the combination therapy reduced the expression of apoptotic cells with only very few apoptotic cells (FIG. 6C). While the kidney sections from AICAR+NAC treated animals did not show any apoptotic cells and these sections appeared similar to control kidney sections (FIG. 6D).

### EXAMPLE 3

### Discussion

Kidneys stored hypothermically for transplantation show varying degrees of tissue injury, depending upon the duration of preservation. The preservation or reversal of damage to organs stored for long periods may be possible by suppressing the factors affecting acute tubular necrosis (ATN), delayed graft functions (DGF) or the loss of organ viability. This may be accomplished either by the development of improved storage methods or by the use of a combination of pharmacologic agents during reperfusion to suppress reperfusion-induced injury. The inventor has already shown the efficacy of a combination therapy in attenuating injury in a rat model (Dobashi et al., 2002) as well as a canine model of normothermic renal ischemic/reperfusion (Sekhon et al., 2003b). The current study shows that a combination therapy of AICAR+NAC attenuates the renal ischemia/reperfusion injury in a canine model of autologous renal transplantation after cold preservation of the kidney for 48 hr. This study indicates that drugs that attenuate various critical cellular events induced by ischemia/reperfusion in different parts of the cell and in different cell types in an organ may provide effective therapy.

The rationale for designing a combination therapy is based on the fact that multiple deleterious events get initiated during ischemia/reperfusion injury in different cell types of an organelle and all of them synergistically contribute towards an irreversible injury. NAC is an antioxidant that reacts with hydroxyl radical and hypochloric acid but is poorly reactive with hydrogen peroxide and the superoxide radical. NAC has been used in a variety of disease states like acetaminophen toxicity, pulmonary oxygen toxicity and human immunodeficiency virus infection. It has been shown the protective effect of pre-administration of NAC on focal cerebral ischemia in rats (Sekhon et al., 2003a).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 5,912,019

U.S. Pat. No. 5,696,109

Alkhulaifi and Pugsley, Br. Heart J., 73:304-305, 1995.

Belzer et al., Lancet., 2:536-538, 1967.

Bird et al., Pharmacology, 50:9-23, 1995.

Blazquez et al., FEBS Lett., 489:149-153, 2001.

Bo et al., Ann. Neurol. 36:778-786, 1994.

Bonfoco et al., Proc. Natl. Acad. Sci. USA, 92:7162-7166, 1995.

Bullough et al., J. Clin. Invest., 94:1524-1532, 1994.

Chiueh et al. Synapse 11:346-348, 1992.

Collins et al., Lancet., 2:1219-1222, 1969.

Culmsee et al., J. Mol. Neurosci., 17:45-58, 2001.

Dawson et al., Proc. Natl. Acad. Sci. USA, 88:6368-6371, 1991.

Dobashi et al., Mol. Cell Biochem., 240:9-17, 2002.

Galinanes et al., J. Thorac. Cardiovasc. Surg., 110:752-761, 1995.

Hagar il., 271:F209-215, 1996.

Herskowitz et al., Am. J. Pathol., 146:419-428, 1995.

Jaffrey et al., Annu. Rev. Cell Dev. Biol., 11:417-440, 1995.

Jamieson et al., Cryobiology, 25:300-310, 1988.

Koeppel et al., Eur. Surg. Res., 28:270-277, 1996.

Koning et al., Transplantation, 63:1620-1628, 1997.

Koprowski et al., Proc. Natl. Acad. Sci. USA, 90:3024-3027, 1993.

Lopez-Neblina et al., Transplantation, 61:179-183, 1996.

Mangano, J. Card. Surg., 10:366-368, 1995.

Matas et al., Transplantation, 69:54-58, 2000.

Mathew et al., J. Thorac. Cardiovasc. Surg., 109:448-456, 1995.

Menasche, Curr. Opin. Cardiol., 10:597-604, 1995.

Merrill et al., J. Immunol., 151:2132-2141, 1993.

Mitrovic et al., Neurosci., 61:575-585, 1994.

Nathan, FASEB J., 6:3051-3064, 1992.

Ojo et al., Transplantation, 63:968-974, 1997.

Page et al., Adv. Exp. Med. Biol., 370:353-356, 1994.

Pahan et al., Free Radic. Biol. Med., 24:39-48, 1998.

Ploeg et al., Transplantation, 46:191-196, 1988.

Remington's Pharmaceutical Sciences 8th and 15th Editions, 1990.

Sekhon et al., Brain Res., 971:1-8, 2003a.

Sekhon et al., J. Nephrol., 16:63-74, 2003b.

Singh et al., J. Biol. Chem., 273:20354-20362, 1998.

Stefanelli et al., Biochem. Biophys. Res. Commun., 243:821-826, 1998.

Takada et al., J. Clin. Invest., 99:2682-2690, 1997.

Tejani et al., Pediatr. Transplant., 3:293-3000, 1999.

Vedder et al., Proc. Natl. Acad. Sci. USA, 87:2643-2646, 1990.

Vemulapalli et al., Pharmacology, 47:188-193, 1993.

Wahlberg et al., Transplantation, 43:5-8, 1987.

Wahlberg et al., Cryobiology, 23:477-482, 1986.

I claim:

1. A method for reducing ischemic/reperfusion injury to an organ in a patient comprising administering a therapeutically effective amount of 5-aminoimmidazole-4-carboxamide ribonucleoside (AICAR) and N-acetyl cysteine (NAC) to the patient.

2. The method of claim 1, wherein the organ is transplanted into the patient.

3. The method of claim 2, wherein AICAR is administered to the patient before, during or after the organ is transplanted into the patient.

4. The method of claim 3, wherein AICAR is administered to the patient before, during and after the organ is transplanted into the patient.

5. The method of claim 2, wherein NAC is administered to the patient before, during or after the organ is transplanted into the patient.

6. The method of claim 5, wherein NAC is administered to the patient before, during and after the organ is transplanted into the patient.

7. The method of claim 2, wherein AICAR is administered to the patient, before, during or after NAC is administered to the patient.

8. The method of claim 2, wherein AICAR is administered to the patient from about 30 min to about 90 min before the organ is transplanted.

9. The method of claim 2, wherein NAC is administered to the patient from about 30 min to about 90 min before the organ is transplanted.

10. The method of claim 1, wherein the organ is a heart, kidney, liver, pancreas or brain.

11. The method of claim 10, wherein the organ is a kidney.

12. The method of claim 1, wherein from about 1 to about 100 mg/kg body weight of AICAR is administered to the patient.

13. The method of claim 1, wherein from about 1 to about 200 mg/kg body weight of NAC is administered to the patient.

14. A method for reducing the risk of organ failure caused by ischemic/reperfusion injury in a patient comprising administering a therapeutically effective amount of 5-aminoimmidazole-4-carboxamide ribonucleoside (AICAR) and N-acetyl cysteine (NAC) to the patient.

15. A method of reducing delayed graft function of a transplanted organ in a patient comprising administering a therapeutically effective amount of 5-aminoimmidazole-4-carboxamide ribonucleoside (AICAR) and N-acetyl cysteine (NAC) to the patient.

16. The method of claim 15, wherein the delayed graft function is caused by acute tubular necrosis.

17. A pharmaceutically acceptable composition comprising 5-aminoimmidazole-4-carboxamide ribonucleoside (AICAR) and N-acetyl cysteine (NAC).

18. The method of claim 17, wherein the first pharmaceutical composition comprises from about 1 to about 100 mg/kg body weight of AICAR.

19. The method of claim 17, wherein the pharmaceutical composition comprises from about 1 to about 200 mg/kg body weight of NAC.
